(19) United States
(12) Patent Application Publication
(10) Pub. No.: US 2025/0089783 A1
(43) Pub. Date: Mar. 20, 2025

(54) VAPORIZER SYSTEM

(71) Applicant: Rite LLC, Benton Harbor, MI (US)

(72) Inventors: Neal A. Beshears, Anderson, IN (US); Hayden B. Keiser, Benton Harbor, MI (US)

(21) Appl. No.: 18/580,356

(22) PCT Filed: Jul. 20, 2022
(86) PCT No.: PCT/US2022/073946

§ 371 Date: Jan. 18, 2024

Related U.S. Application Data
(60) Provisional application No. 63/225,032, filed on Jul. 23, 2021.

(51) Int. Cl. A24F 40/46; A24F 40/10; A24F 40/42; A24F 40/48

(52) U.S. Cl. CPC A24F 40/46; A24F 40/10; A24F 40/42; A24F 40/48

(57) ABSTRACT

A vaporizer system including a portable unit and a tabletop unit is provided. The system can be used in a first mode and in a second mode to vaporize a substance provided to the system by a user. In the first mode, an individual user may inhale vapors generated by the system through a mouthpiece coupled to the portable unit. In a second mode, one user or multiple simultaneous users can inhale the vapors generated by the system via outlets coupled to an aeration chamber of the tabletop unit. The tabletop unit may further include vials that house aromatic substances that can flavor the vapors generated by the system. In some embodiments, the vials may include adjustable apertures that allow the user to change the amount of aromatics provided to the aeration chamber.

### CROSS-REFERENCE TO RELATED APPLICATIONS

This application is a U.S. National Phase Application of International Application No. PCT/US2022/073946, filed Jul. 20, 2022, which claims priority to U.S. Provisional Patent Application Ser. No. 63/225,032, filed on Jul. 23, 2021, entitled “RITE CONVECTION SYSTEM,” the entire disclosure of which is incorporated herein by reference.

### FIELD OF INVENTION

The present invention relates generally to vaporizer systems that enable the inhalation of vapors from heated substances. More specifically, the present invention relates to a vaporizer system that may be used as either a portable or a tabletop device.

### BACKGROUND OF INVENTION

Recent years have seen an upsurge in the use of vaporizers that electronically heat substances to release aerosolized compounds or vapors that can be inhaled by users. Many consumers prefer vaporizers to traditional means of smoking because the vaporizers reduce the risk of combustion during vaporization, which releases compounds that may degrade the taste of the product and harm the consumers' health. Moreover, vaporizers are more convenient for consumers and allow for a more even heating of a given substance, thereby providing a more consistent and enjoyable user experience.

Two distinct categories of vaporizer products have emerged: those designed for individual use, and those designed to be used by a group of individuals simultaneously. Products geared toward group use are larger than products designed for individual use and must be used at a fixed location as they generally require a power source. On the other hand, products geared toward individual use generally take the form of portable, handheld devices which are powered by rechargeable batteries. This bifurcation generates inconvenience and may increase costs for consumers who wish to use a vaporizer product both individually and as part of a group, as the consumers must purchase a separate device for each purpose.

Traditional vaporizers have been developed for use with a variety of substances including herbs, oils, concentrates, and nicotine-containing liquids. These vaporizers are designed for use with various subsets of these substances, but not all of the substances. As a result, customers that wish to use the full range of substances must buy a minimum of two devices which increases cost and generates inconvenience as the two devices may not be compatible with each other.

In addition, traditional vaporizers only utilize a single chamber to hold substances for heating. As a result, consumers may only vaporize one substance at a time. Because of this design limitation, consumers can only enjoy the taste or flavor of one substance at a time. If a consumer desires to experience a new taste or flavor, the consumer must insert an entirely new substance into the vaporizer or physically mix different substances together before inserting them into the vaporizer.

Finally, many products have a rudimentary user interface and do not provide helpful information or other feedback to consumers beyond giving some estimate of the product's current battery life (usually indicated by a single, multi-colored light). Other products only incorporate a small digital interface capable of displaying information regarding the current battery life and wattage or voltage settings of the device, but little else. Thus, these devices give consumers little meaningful insight into the status of the device itself or of the substances stored in the device.

### SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

A vaporizer system is provided comprising a portable vaporizer device (the “portable unit”) and a tabletop device (the “tabletop unit”). These devices enable the vaporizer system to be used in at least a first mode and a second mode. The first mode may be a portable mode wherein the vaporizer system may be used by a single individual in any location, and the second mode may be a tabletop mode wherein the vaporizer system may be used by one or more individuals simultaneously.

The portable unit may include a heating mechanism which can produce vapors from substances including, but not limited to, herbs, oils, concentrates, and nicotine-containing liquids. The portable unit may heat the substances via convection (wherein a heating element heats the air surrounding the substance) or conduction (wherein the substance is heated via direct contact with the heating mechanism). In some embodiments of the invention, the heating element may produce vapors from the substance without burning or combusting the substance.

The portable unit may include a mouthpiece. Different mouthpieces may be used with different substances that can be vaporized by the portable unit, and the mouthpieces can be “swapped out” by the user. The user may activate the portable unit by bringing the mouthpiece to their lips and inhaling at a first rate. In some embodiments, the portable unit may provide a feedback signal (e.g., a blinking light or a vibration) to notify users that it has been powered on.

Users may select a temperature used to vaporize the substance by, for example, shaking the portable unit or by using a mobile application which connects to the portable unit via a wireless connection. Once the desired temperature is selected, the user may initiate the heating process by inhaling through the mouthpiece at the first rate of inhalation. The portable unit may again trigger the feedback signal when the heating process is initiated.

When the heating process is complete, the feedback signal may notify the user that the device has reached the desired heating temperature. The user may then inhale the vapors that are released from the substance by bringing the portable unit to their lips and inhaling at a second rate. In some embodiments, the second rate of inhalation may be greater than the first rate of inhalation.

When in the second mode, the portable unit may be “plugged into” or coupled to the tabletop unit. When retained in the tabletop unit, the portable unit may be in electrical communication with a source of power (e.g., a battery housed in the tabletop unit or an electrical outlet) which may provide power to the portable unit to recharge the portable unit. In the second mode, the vapors produced by the portable unit may travel through a first, portable conduit to an aeration chamber in the tabletop unit. From the aeration chamber, vapors may be inhaled by one or more users via one or more outlets in fluid communication with the aeration chamber. More specifically, the user or users may couple a tube or hose to an outlet of the one or more outlets to inhale the vapors generated by the vaporizer system. Advantageously, multiple users may simultaneously inhale the vapors from the vaporizer system at the same time.

In addition, the tabletop unit may be coupled to one or more containers or vials. Each vial may contain an aromatic substance (e.g., loose-leaf botanicals or oils) and may include a humidity gauge or a humidity sensor that may assist the user in monitoring the health of the aromatic substance retained within each vial. The aromatic substances retained in the vials produce aromatic vapors which may travel to the aeration chamber via a second, vial conduit. Within the aeration chamber, the vapors produced by the portable unit's heating mechanism and the aromatic air from the vials may combine into a single airstream to be inhaled by the users, thereby enhancing the flavor of the vapors. In some embodiments of the system, the vials may be coupled to the tabletop unit via a pivoting mechanism. The pivoting mechanism may allow the vials to swing upwardly and away from the surface of the tabletop unit such that the users may open the vials and add or remove aromatic substances from the vials.

In addition, the user may selectively adjust the flavor profile of the vapors produced by the tabletop unit by adjusting the amount of aromatic vapors provided to the aeration chamber. In a preferred embodiment, the user may adjust the size of an aperture provided within the vials to control the amount of aromatic vapors flowing out of the vials. For example, if the user wishes to increase the amount of aromatic vapors provided from the vials to the aeration chamber, the user may increase the size of one or more of the apertures within the vials. Advantageously, because the user may control the amount of aromatic vapors flowing out of each vial, and each vial may house a different aromatic substance, the user may create a nearly limitless combination of flavor profiles.

In some embodiments, the tabletop unit may also include a digital interface. The digital interface may provide users with information such as the battery life of the portable unit, the battery life of the tabletop unit, the selected heating temperature, the time, the system's wireless connectivity status, and the health of the aromatic substances stored in the vials (as indicated by the humidity gauge or gauges).

### BRIEF DESCRIPTION OF DRAWINGS

For a better understanding of the various embodiments of the present invention, reference may be made to the accompanying drawings in which:

FIG. 1 is an isometric view of a vaporizer system comprising a portable device and a tabletop device according to an example embodiment;

FIG. 2 is an isometric view of the portable device of FIG. 1 separated into a top portion and a bottom portion;

FIG. 3 is an exploded view of the top portion of FIG. 2;

FIG. 4 is an exploded view of the bottom portion of FIG. 2;

FIG. 5 is an exploded view of a second embodiment of the portable device of FIG. 1;

FIG. 6 is a plan view of the vaporizer system of FIG. 1; and

FIG. 7 is a partial cross-sectional view of vials of the tabletop unit of FIG. 1.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION OF THE INVENTION

In the following description, various embodiments of the present invention will be described. For purposes of explanation, specific configurations and details are set forth in order to provide a thorough understanding of the embodiments. It will also be apparent to one skilled in the art, however, that the present invention may be practiced without the specific details. Furthermore, well-known features may be omitted or simplified in order not to obscure the embodiment being described.

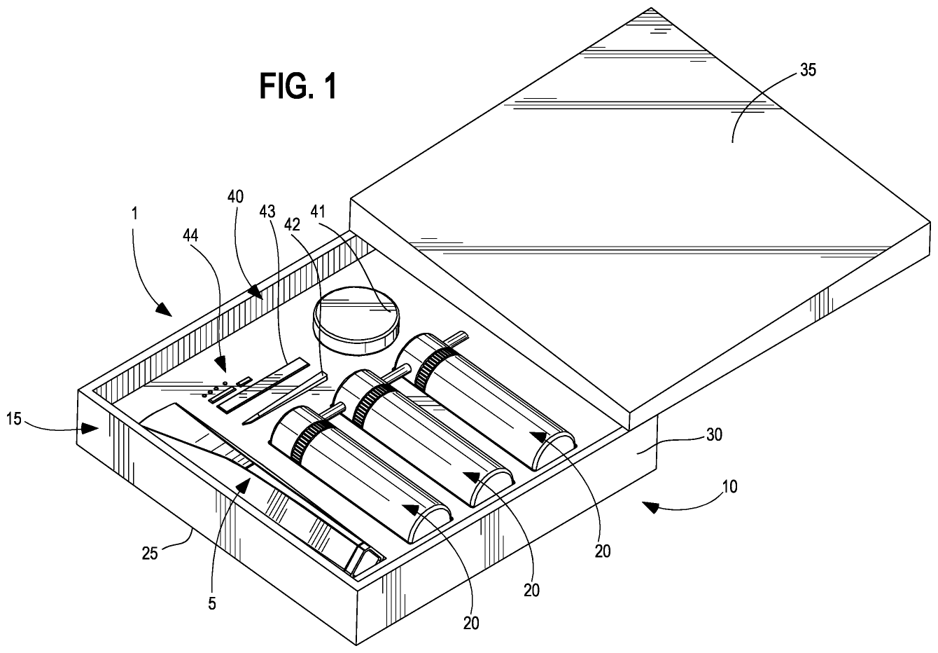

FIG. 1 illustrates a vaporizer system 1 (hereinafter, the “system 1”) that may be used by an individual or a group of people to inhale vapors generated by the system 1. Generally, the system 1 may be used in a first mode or a second mode. In the first mode, an individual user may utilize a portable unit 5 to inhale vapors generated by the portable unit 5. In the second mode, the vapors generated by the portable unit 5 may be provided to at least one user via a tabletop unit 10. If desired, multiple users can inhale vapors generated by the system 1 in the second mode simultaneously. The tabletop unit 10 may include a body 15 which is configured to retain the portable unit 5 and at least one vial 20. The portable unit 5 and the at least one vial 20 may be in fluid communication such that any aromatic substances retained within the vials 20 can flavor the vapors produced by the portable unit 5, if such flavoring is desired by the user. Each vial 20 may further include a humidity sensor (not illustrated) that allows the user to monitor the freshness of the aromatic substances contained or stored within the vial 20. The humidity sensor may help users determine when it is appropriate to replace the aromatic substances with fresh aromatic substances.

When replacing the aromatic substances of the at least one vial 20, the user may decouple the at least one vial 20 from the body 15 and open the at least one vial 20. Alternatively, the at least one vial 20 may be coupled to a pivoting mechanism that allows a portion of the at least one vial 20 to pivot upwardly and away from the body 15. When the at least one vial 20 pivots away from the body 15, the user may open the at least one vial 20 and access its interior contents.

The body 15 of the tabletop unit 10 may be provided in the form of a rectangular prism, although other shapes are foreseeable. The body 15 may include at least one surface, such as a bottom surface 25, which is substantially flat to help the tabletop unit rest stably on another surface (e.g., a floor, a desk, a table, and a counter). The body 15 may also comprise at least two components, specifically a bottom portion 30 and a lid 35, such that the tabletop unit 10 may be closed when the tabletop unit 10 is not in use. The lid 35 may be substantially the same size as, or slightly larger than, the bottom portion 30 such that the lid 35 may be placed over the bottom portion 30. In addition, the lid 35 may be coupled to the bottom portion 30 or provided as a separate piece. In some embodiments, the lid 35 may further include a tray upon which the user may prepare substances for use within the system 1.

The tabletop unit 10 may also include various accessories 40 that may add functionality to the system 1. The accessories 40 may include items such as a grinder 41, a pick 42, and a scraper 43 that can assist a user in preparing a substance for vaporization and/or be used for cleaning the system 1. For example, if the user desires to vaporize an herb, the grinder may be used to crush the herb into smaller pieces. In addition, the accessories 40 may further include a human-machine interface 44 that may allow the user to control the operation of the system 1, as desired. Various uses for the accessories 40, and other accessories that may be provided in conjunction with the system 1, would be known and appreciated by those skilled in the art.

The tabletop unit 10 may be powered by an internal battery and/or via an external energy source such as a wall outlet. This may allow the tabletop unit 10 to provide power to various electronic components, including the portable unit 5 when the portable unit 5 is received within the tabletop unit 10. In turn, the portable unit 5 may use power from the tabletop unit 10 to vaporize the substance or recharge a battery housed within the portable unit 5.

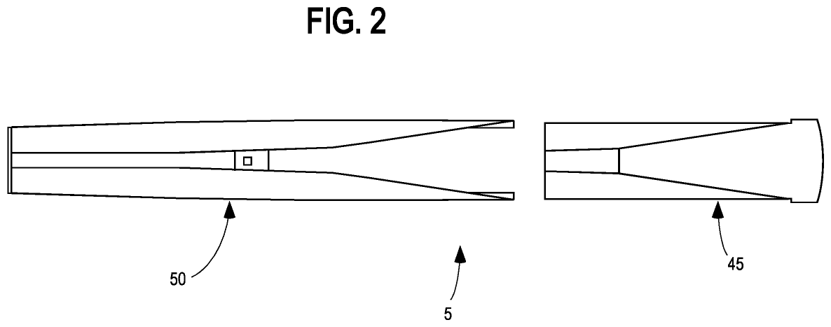

Turning to FIG. 2, the portable unit 5 may further include a top portion 45 and a bottom portion 50. The portions 45, 50 may be coupled to one another via a friction fit, a hook and tab mechanism, magnets, or other attachment means that would be appreciated by those skilled in the art. When the portions 45, 50 are decoupled (as illustrated), the user may access internal components of the portable unit 5 and/or insert into the portable unit 5 the substance that will be vaporized. As understood by those skilled in the art, the substance that can be provided to the portable unit 5 include, but is not limited to, herbs and other plant-derived matter, oils, concentrates, and the like.

Each of the portions 45, 50 may house components of the portable unit 5, including, but not limited to, magnets, indicator lights, speakers, wireless transmitters, wireless receivers, sensors, heating elements, heating chambers, substance chambers, heat sinks, filters, batteries, printed circuit boards, and/or vapor conduits. Finally, either of the portions 45, 50 may serve as a mouthpiece via which the user can inhale vapors generated by the portable unit 5.

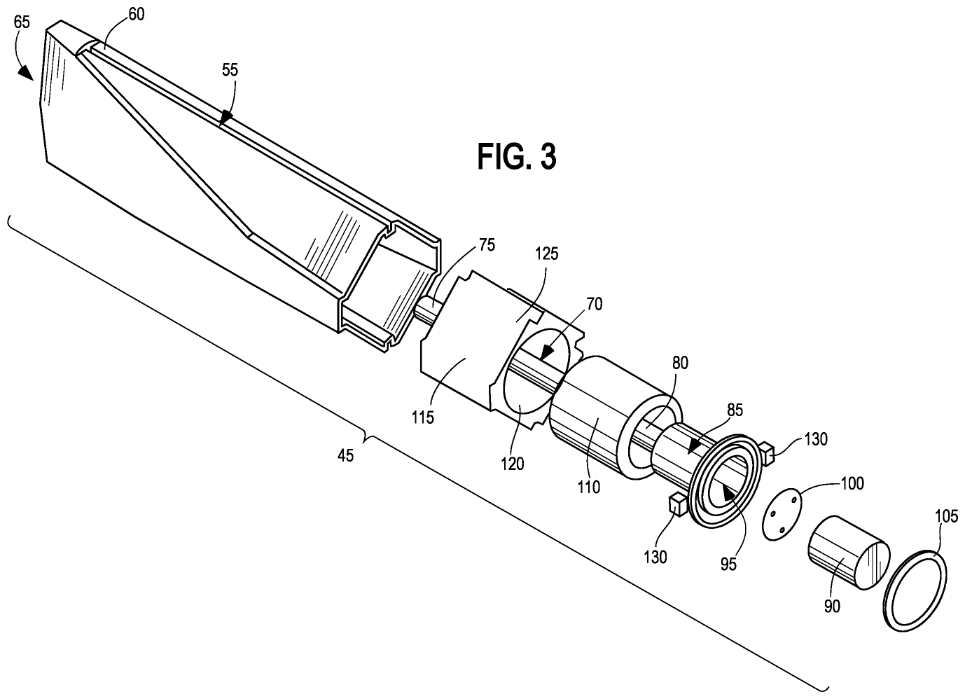

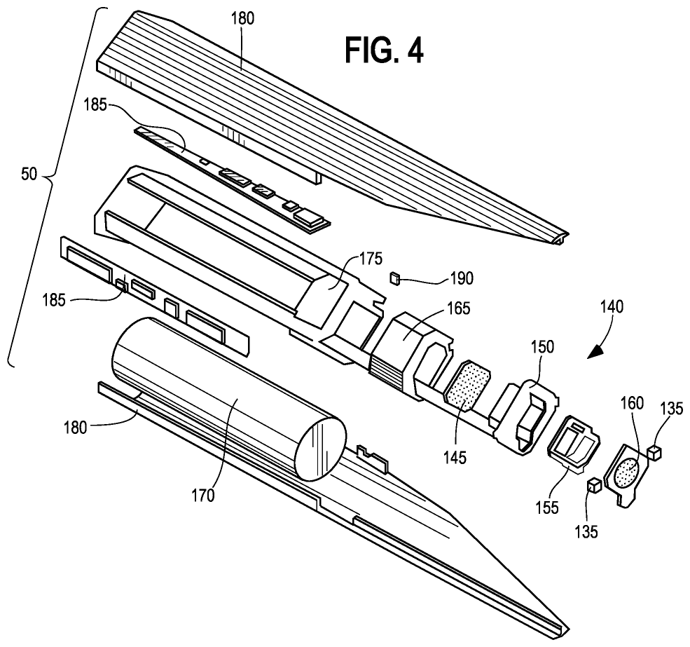

Together, FIGS. 3 and 4 illustrate exploded views of one embodiment of the portable unit 5. Although FIGS. 3 and 4 depict various components as being retained within either the top portion 45 or the bottom portion 50, one skilled in the art would appreciate that in other embodiments of the invention the components may be located in either of the portions 45, 50.

As illustrated in FIG. 3, the top portion 45 may include a cover 55 provided in the shape of a rectangular prism with at least one open end, although other shapes are foreseeable. The cover 55 may be composed of a durable material (e.g., plastic or aluminum) to help protect the components retained therein. A first end 60 of the cover 55 may function as a mouthpiece 65 through which the user may inhale vapors generated by the portable unit 5.

The top portion 45 may further include a pipe 70 comprising a first end 75 that is in fluid communication with an opening (not illustrated) in the mouthpiece 65. Thus, the pipe 70 may provide a conduit by which vapors generated by the portable unit 5 can be provided to the user. The pipe 70 may be provided as an open, elongated cylinder composed of corrosion resistant materials (e.g., stainless steel). A second end 80 of the pipe 70 may be coupled to, or provided as, a chamber 85 that can retain a substance 90 that a user desires to vaporize with the portable unit 5. The chamber 85 may be provided as a three-dimensional body with a void 95 within which the substance 90 can be retained during the vaporization process.

The identity of the substance 90 is not particularly limited, and may include oils, herbs, concentrates, and other compounds and materials provided in a solid or liquid form. If the substance 90 is provided in a liquid form, the substance 90 may be retained within a vessel (not illustrated) that can be inserted into the chamber 85 by the user. No matter the identity of the substance 90, the vapors generated from the substance 90 during the vaporization process may be drawn through the pipe 70 and inhaled by the user.

Optionally, or preferably, a filter 100 may be provided within the chamber 85 to filter particulates or other unwanted components produced during vaporization of the substance 90, which in turn may create a more pleasant experience for the user. The filter 100 may be positioned and located between the substance 90 and the first end 75 of the pipe 70 such that the vapors pass through the filter 100 before being inhaled by the user. The pore size of the filter 100 is not limited, and the user may select from a variety of filters that are known in the art.

The chamber 85 may be coupled to a gasket, a magnet, or a member 105, which may help seal the chamber 85 when it is coupled to a heating mechanism (see FIG. 4). The member 105 may help prevent vapors from leaking into an interior volume of the portable unit 5 during the vaporization process.

To help prevent the components or outside surfaces of the portable unit 5 from overheating during the vaporization process, an insulation body 110 may be provided. The insulation body 110 may be composed of a material with a low thermal conductivity (e.g., fiberglass) such that the vaporization process is substantially retained in the chamber 85 or is slowly dissipated from the chamber 85. Thus, the insulation body 110 may help prevent components of the portable unit 5 from melting and/or help prevent the user from being burned during the vaporization process. The insulation body 110 may be provided as an open cylinder that is slightly larger than the chamber 85, although other shapes and sizes for the insulation body 110 are foreseeable. Preferably, the insulation body 110 is positioned and located such that it substantially surrounds the chamber 85.

A support 115 may be provided in the top portion 45 to help secure the insulation body 110, the chamber 85, and other components within the top portion 45. The support may be a three-dimensional structure provided in substantially the same shape as the cover 55, although other shapes for the support 115 are foreseeable. The support 115 may include a bore 120 that extends through a body 125. The insulation body 110 and/or the chamber 85 may be retained within the bore 120 via a friction fit or other means that would be appreciated by those skilled in the art.

The top portion 45 may further include a magnet or magnets 130 positioned and located on the chamber 85, although other locations for the magnets 130 are foreseeable. The magnets 130 may help selectively couple the portions 45, 50 together via a magnetic force. For example, the magnetic force may be generated via an attraction between the magnets 130 and a metallic component of the bottom portion 50. Alternatively, such as in the embodiment illustrated in FIG. 4, the magnetic force may be generated via an attraction between the magnets 130 and a magnet or magnets 135 provided with the bottom portion 50.

The bottom portion 50 may include a heating mechanism 140 comprising a heating element 145, a heating vessel 150, a heat sink 155, and a filter 160. Generally, the heating mechanism 140 may interface with the chamber 85 such that the substance 90 can be vaporized when it is retained within the chamber 85. The heating element 145 may be provided as a resistor or a ceramic rod that is retained within the heating vessel 150, although other heating mechanisms are known in the art. The heating vessel 150 may be substantially surrounded by an insulation body 165. The insulation body 165 may be composed a material with a low thermal conductivity (e.g., fiberglass) such that the heat generated by the heating element 145 is substantially retained within the heating vessel 150 and/or provided to the chamber 85. The filter 160 may help prevent portions of the substance 90 from falling into the heating mechanism 140 and/or the heating vessel 150. The pore size of the filter 160 is not limited, and the user may select from a variety of filters that are known in the art.

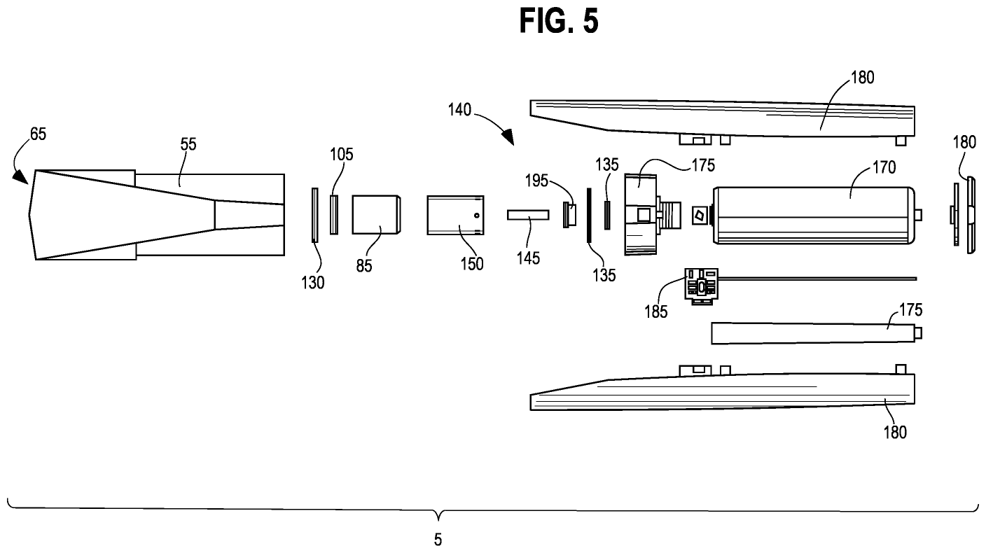

The heating element 145 may be adapted to heat the substance 90 retained in the chamber 85 via conduction and/or convection. For example, the heating element 145 may contact the heat sink 155, the substance 90, and/or the chamber 85 to supply heat energy to the substance 90, which in turn may vaporize the substance 90. As an additional example, if the substance 90 is an oil provided in a vessel, the heating element 145 may contact the vessel holding the substance 90 such that heat energy is transferred to the substance 90. As a further example, the heating element 145 may heat the surrounding air and provide the heated air to the substance 90 to vaporize the substance 90. If the substance 90 is provided as an herb, the convection process may first remove moisture from the herb and then vaporize the substance 90 with reduced or no combustion. In some embodiments, the heating mechanism 140 may provide energy to the heating element via a magnetic or induction heating process, as explained in more detail with reference to FIG. 5 below.

The portable unit 5, and the various electronic components thereof, may be powered by a battery 170. Preferably, the battery 170 may be provided as a lithium-ion battery, a nickel-cadmium battery, or another rechargeable battery known in the art. In some embodiments, the battery 170 may also be in electrical communication with a port or an inlet (not illustrated) via which electrical energy can be provided to the portable unit 5 to recharge the battery 170. In other embodiments of the invention, the battery 170 may be recharged via a wireless charging connection.

The bottom portion 50 may include a frame 175 to help secure the various components of the bottom portion 50. The frame 175 may be composed of a durable material (e.g., metal or plastic) that can provide structural support to the components of the portable unit 5. For example, the heating mechanism 140, the magnets 135, the insulation body 165, and/or additional components of the bottom portion 50 may be retained within and/or coupled to the frame 175. The frame 175 may be provided as a hollow, three-dimensional rectangular structure, although other shapes for the frame 175 are foreseeable. The frame 175 may be the substantially the same size as, or smaller than, a cover portion 180 and may be coupled to the cover portion 180 via an attachment means or a friction fit.

The frame 175 may also retain or be coupled to at least one printed circuit board 185 (hereinafter, the PCB 185). The PCB 185 may be coupled to the frame 175 via an attachment means (e.g., glue or epoxy). The PCB 185 may be in electrical communication with the battery 170 and may include programmable circuits, microprocessors, sensors, timers, mechanical actuators, wireless transmitters, wireless receivers, wired transmitters, wired receivers, and the like. The PCB 185 may provide additional functionality for the portable unit 5 and/or the system 1. For example, the PCB 185 may include programming that allows the user to select the heating temperature of the heating element 145. As an additional example, the PCB 185 may include a timer that “times out” and deactivates the portable unit 5 after a predetermined amount of time in order to conserve the battery 170. As a further example, the PCB 185 may communicate wirelessly with the tabletop unit 10 and/or a remote device such as a cell phone or laptop computer (not illustrated). As yet another example, the PCB 185 may further include and/or be in communication with sensors including flowmeters that can detect the rate of airflow through the mouthpiece 65 or the interior of the portable unit 5.

The PCB 185 may further be in communication with a signal 190. The signal 190 may be provided as a component (e.g., a light, a speaker, a mechanical actuator, or combinations thereof) that may, for example, change color, change brightness, blink, play various sounds, or vibrate in response to predetermined actions or events. More specifically, in some embodiments of the invention, the signal 190 may: light up when the user activates the portable unit 5; vibrate when the heating element 145 is activated; and/or change its brightness when the heating element 145 reaches a predetermined or desired temperature.

A method of using the system 1 in the first mode is also provided. To begin, the user may select the substance 90 the user desires to vaporize. The user may then decouple the portions 45, 50 from one another and place the substance 90 into the chamber 85. After recoupling the portions 45, 50, the user may activate the portable unit 5 by inhaling through the mouthpiece 65 at a first rate, which may also trigger the signal 190. Once the portable unit 5 is activated, the user may select the desired heating element temperature by moving or shaking the portable unit 5. After the desired temperature is selected, the user may initiate the heating process by again inhaling through the mouthpiece 65 at the first rate, or the heating element 145 may activate automatically after a predetermined amount of time.

When the heating element 145 is activated, the signal 190 may be triggered. After the desired heating temperature is achieved, the signal 190 may again be activated. The user can then inhale vapors generated by the portable unit 5 by inhaling through the mouthpiece 65 at a second rate. Preferably, the second rate is equal to or greater than the first rate. When the user wishes to deactivate the portable unit 5, the user may move or shake the portable unit or let a predetermined amount of time elapse without inhaling through the mouthpiece at the first rate or the second rate.

In alternative embodiments of the above-described method, the user may activate the portable unit 5 via a push-button mechanism. In other embodiments, the temperature of the heating element 145 may be selected via a physical mechanism (e.g., push buttons or a rotatable dial) or via the remote device wirelessly connected to the portable unit 5. While the foregoing has been provided as a representative case of how the user may utilize the system 1 in the first mode, one skilled in the art would appreciate that the method may comprise fewer steps or additional steps than those described above.

FIG. 5 illustrates a second example embodiment of the portable unit 5 that may have substantially the same functionality as the portable unit 5 provided in FIGS. 2-4. This second embodiment comprises many of the components of the portable unit 5 discussed above and further includes a heating mechanism 140 that may operate via an electromagnetic or an induction heating process. In this embodiment, an induction member 195 may, through known mechanisms, provide energy to the heating element 145 via magnetic waves. In turn, this energy may cause the temperature of the heating element 145 to increase. The heating element 145 may then provide this energy, via conduction or convention, to the substance 90 retained within the chamber 85 and/or the heating vessel 150. As a non-limiting example, the heating element 145 may heat the air retained within the heating vessel 150. As the air warms within the heating vessel 150, convection currents may transport the heat energy to the substance 90, vaporizing the substance 90. The vapors produced from the vaporization of the substance 90, and any residual heat energy, may then exit the portable unit 5 via the mouthpiece 65.

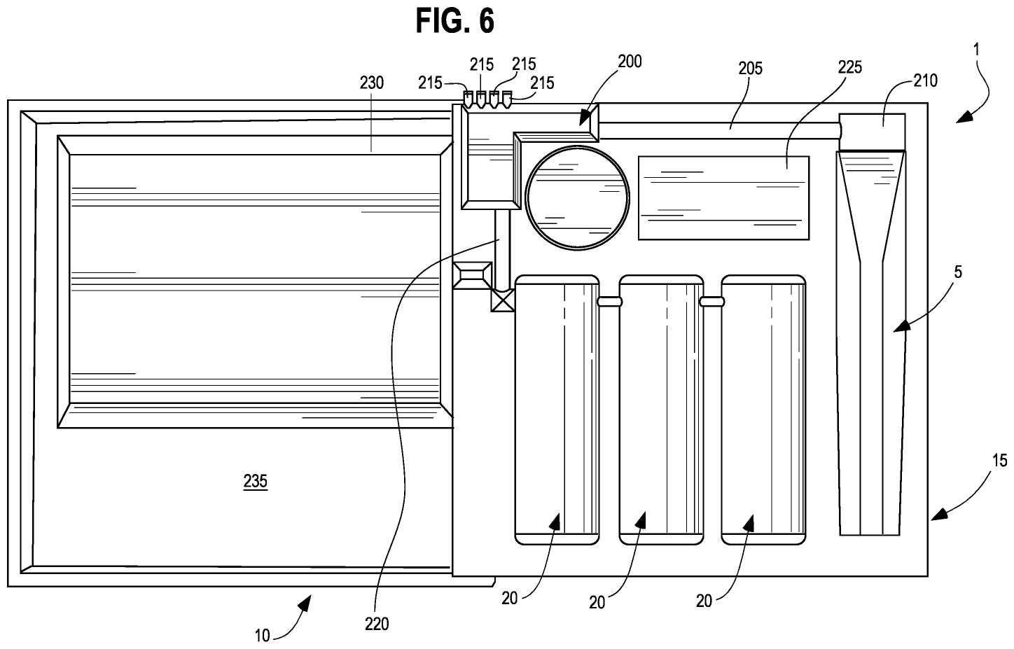

Turning to FIG. 6, the portable unit 5 is in fluid communication with an aeration chamber 200 of the tabletop unit 10 via a conduit 205. The conduit 205 may include a port 210 that interfaces with the mouthpiece 65, although in other embodiments the conduit 205 may directly couple to the portable unit 5. When the system 1 is used in the second mode, vapors produced by the portable unit 5 may flow to the aeration chamber 200 via the conduit 205 and/or the port 210. The vapors generated by the portable unit 5 may flow into the aeration chamber 200 via the action of diffusion and/or the inhalation of the user or users of the system 1.

The aeration chamber 200 may be provided as a three-dimensional body with an open interior adapted to retain gases and vapors produced by the system 1. The aeration chamber 200 may include at least one outlet 215 positioned and located such that the at least one outlet 215 points outwardly and away from the body 15. Here, the tabletop unit is provided with four outlets 215, although a tabletop unit with a different number of outlets 215 is also foreseeable. A hose, tubing, or the like (not illustrated) may be coupled to each outlet 215. When the user inhales through the hose or tubing, the user can draw vapors out of the system 1 via the aeration chamber 200 and into the hose or tubing. In embodiments including more than one outlet 215, more than one user may inhale the vapors produced by the system 1 simultaneously.

Advantageously, the vapors produced by the system 1 may be flavored with aromatic vapors or aromatic gases provided from the vials 20. Each vial 20 retained in the tabletop unit 10 may be in fluid communication with the aeration chamber 200 via a conduit 220. The vials 20 may each contain an additional substance that generates the aromatic vapors. The user may load each vial 20 with identical aromatic substances or different aromatic substances as desired. Ultimately, the aromatic vapors may be provided to the aeration chamber 200 via diffusion and/or the inhalation of the user or users. The aromatic substance itself may be an oil, a concentrate, an herb, or the like that can produce aromatic vapors with the use of or without the use of a heating element.

The conduits 205, 220 may further include check valves, one-way flow valves, or the like to help ensure that the vapors produced by the portable unit 5 and/or the aromatic vapors provided by the vials 20 substantially flow in one direction. For example, the aforementioned valves may help ensure that the vapors and the aromatic vapors flow from their source to the aeration chamber 200. In addition, the outlets 215 may be provided as one-way flow valves such that air from the environment outside of the system 1 does not enter the aeration chamber 200 via the outlets 215.

The tabletop unit 10 may further include a display 225. The display 225 may be adapted to display information related to operational parameters of the system 1, including the temperature of the heating element 145, the temperature of the substance 90, the humidity within the vials 20, the battery life of the portable unit 5, the battery life of the tabletop unit 10, the wireless connectivity status of the system 1, and other similar parameters. In addition, the display 225 may display information unrelated to the operational parameters of the system 1, such as the local time. Furthermore, the display 225 may be provided as a touchscreen such that the user may input commands to control various operational parameters of the system 1, including (but not limited to) the temperature of the heating element 145.

In some embodiments, the tabletop unit 10 may include a transmitter and a receiver such that the system 1 may communicate with a remote device (e.g., a laptop computer or a cell phone, not illustrated). The transmitter and the receiver may be provided in a wired or wireless form. In such embodiments, information regarding operational parameters of the system 1 may be displayed on a screen of the remote device. In addition, the user may alter the operational parameters of the system 1 via the remote device, such as the temperature of the heating element 145.

The tabletop unit 10 may further include a tray 230 that can provide the user with a convenient location for preparing substances before they are used with the system 1. The tray 230 may be provided in the shape of a rectangular prism, although other shapes for the tray 230 are foreseeable. The tray 230 may be provided as a component that couples to a surface of the lid 35, (e.g., a surface 235) or the tray 230 may be provided as a raised protrusion that projects outwardly from the lid 35. The tray 230 may magnetically couple to the lid 35, although other coupling mechanisms would be appreciated by those skilled in the art.

In some embodiments, the tabletop unit 10 may further include a support structure (not illustrated) that may provide support to the body 15 and/or the lid 35 when the tabletop unit 10 is open. The support structure may be provided as a kickstand or a leg member that holds the body 15 and/or the lid 35 in position during use of the tabletop unit 10. For example, the support structure may extend downwardly from the lid 35 and abut a surface upon which the tabletop unit 10 is positioned such that the lid 35 may be held above the surface. Other configurations for the support structure would be appreciated by those skilled in the art.

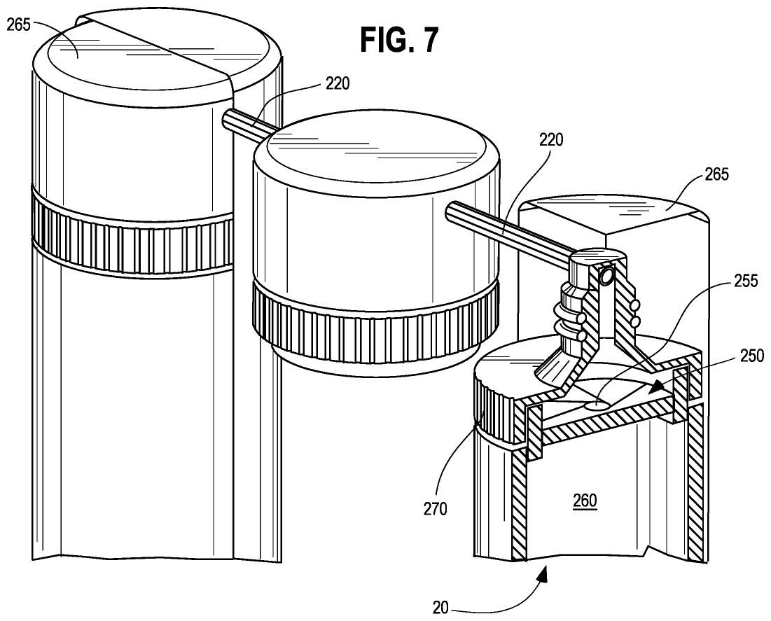

As illustrated in FIG. 7, the vials 20 may include a selectively adjustable mechanism 250 that can be placed in an open position or a closed position. Functionally, the mechanism 250 may allow the user to control the amount of aromatic vapors introduced to the aeration chamber 200 from the vials 20. The mechanism 250 may include an opening or an aperture 255 whose size can selectively be altered by the user. When the mechanism 250 is in the closed position, the aperture 255 is closed and the aromatic vapors within an interior 260 of the vial 20 are substantially prevented from travelling to the aeration chamber 200. In comparison, when the mechanism 250 is in the open position and the aperture 255 is open, the interior 260 is in fluid communication with the conduit 220 and the aeration chamber 200. Advantageously, the size of the aperture 255 may be selectively adjusted by the user to change the amount of aromatic vapor supplied to the aeration chamber 200. For example, if the size of the aperture 255 is increased, more aromatic vapors may diffuse into or be pulled into the aeration chamber 200 as compared to situations where the aperture 255 is provided as a smaller size.

The user may change the size of the aperture 255 by twisting a cap 265 of the vial 20 and/or a ring 270 positioned and located on the cap 265 in a first direction or a second direction. In one embodiment, to place the mechanism 250 in the closed position, the user may twist the cap 265 of the vial 20 in a first direction until the aperture 255 is closed. The user may open the mechanism 250 by twisting the cap 265 in a second direction, which increases the size of the aperture 255. If the user continues to twist the cap 265 in the second direction, the size of the aperture 255 may continue to increase until the aperture 255 reaches its maximum size. Preferably, the width of the aperture 255 when the aperture 255 is at its maximum size is the same as, or slightly smaller than, the width of the vial 20.

By changing the size of the aperture 255, the user may utilize the tabletop unit 10 to create combinations of custom flavors of vapors for inhalation. For example, the user may increase the size of one aperture 255 or multiple apertures 255 to increase the amount of aromatic vapors provided to the aeration chamber 200, thereby changing the flavor profile of the vapors inhaled by the user. Further, because each vial 20 may contain a different substance and the user can adjust the aperture 255 for each vial, the user can customize the amount of each type of aromatic vapor provided to the aeration chamber 200. In effect, the finely tuned customization provided by the mechanism 250 may allow the user to create a nearly limitless profile of vapor flavors for inhalation.

In some embodiments of the invention, the tabletop unit 10 may include a sensor that detects whether the mechanism 250 is in the open position or the closed position. In turn, this information may be provided to the display 225 and/or the remote device for review by the user. Further, in other embodiments of the invention, opening and closing of the mechanism 250 may be effectuated by commands input into the display 225. In addition, each vial 20 may further include the humidity sensor (not illustrated) that is configured to detect the humidity levels of the interior 260. The measurements from the humidity sensor may be provided to the display 225 and/or the remote device for review by the user.

A method of using the system 1 in the second mode is also provided. To begin, the user may position the tabletop unit 10 on a surface, such as a desk or a table and open the lid 35. Then, the user may remove the portable unit 5 from the tabletop unit 10, insert the substance 90 into the portable unit 5, and recouple the portable unit 5 to the tabletop unit 10. Next, the user may configure the mechanism 250 of each vial 20 to adjust the amount of aromatic vapors each vial 20 provides to the aeration chamber 200. After, the user may couple a hose or tubing to the at least one outlet 215 and inhale through the hose or tubing. The user's inhalation may activate the portable unit 5, specifically the heating mechanism 140 within the portable unit 5. At this time, if the display 225 is provided, information regarding the operational parameters may be provided on the display 225, including the battery life of the tabletop unit 10, the battery life of the portable unit 5, the selected temperature of the heating mechanism 140, whether vials 20 are coupled to the tabletop unit 10, and/or if the mechanisms 250 are in the open position or the closed position. If desired, the user may adjust the selected operational parameters of the system 1 via the display 225 or by manually adjusting the parameters as described previously. After the user selects the desired operational temperature for the heating mechanism 140, the heating process begins and the signal 190 may provide a first signal. When the signal 190 provides a second signal, the heating process is complete, and the user may inhale the vapors generated by the system 1 via the hose or tubing connected to the outlet 215. Finally, if the system detects no inhalation for a predetermined amount of time, the system 1 may power off to conserve energy.

As would be appreciated by those skilled in the art, the method of using the system 1 in the second mode may include any additional steps previously described herein. Further, the method may omit steps as desired by the user.

As is evident from the foregoing description, certain aspects of the present invention is not limited by the particular details of the examples illustrated herein, and it is therefore contemplated that other modifications, applications, variations, or equivalents thereof, will occur to those skilled in the art. Many such changes, modifications, variations and other uses and applications of the present constructions will, however, become apparent to those skilled in the art after considering the specification and the accompanying drawings. In addition, unless mention was made above to the contrary, it should be noted that all of the accompanying drawings are not to scale. All such changes, modifications, variations and other uses in applications which do not depart from the spirit and scope of the present inventions are deemed to be covered by the inventions which are limited only by the claims which follow.

1. A vaporizer system comprising:
a portable unit including
a heating mechanism; and
a chamber in communication with the heating mechanism;
a tabletop unit including
a body;
at least one outlet extending from the body; and
at least one conduit couplable to the portable unit when the portable unit is received within the body;
wherein the at least one conduit places the chamber of the portable unit and the at least one outlet in fluid communication; and
wherein the tabletop unit is adapted to provide vapor to at least one user via the at least one outlet.

2. The vaporizer system of claim 1, wherein the vaporizer system operates in at least a first mode and a second mode.

3. The vaporizer system of claim 2, wherein the first mode allows an individual user to intake vapors through a mouthpiece of the portable unit and the second mode allows the at least one user to inhale the vapors via the tabletop unit.

4. The vaporizer system of claim 1, wherein the heating mechanism heats a substance retained within the chamber of the portable unit in both a first mode and a second mode.

5. The vaporizer system of claim 1, wherein the chamber is adapted to retain a substance comprising at least one of an herb, an oil, a concentrate, and an aromatic.

6. The vaporizer system of claim 1, wherein the tabletop unit further includes at least one vial that can be selectively inserted into the body of the tabletop unit.

7. The vaporizer system of claim 6, wherein the at least one vial further includes a humidity sensor adapted to monitor a humidity of the at least one vial.

8. A vaporizer system comprising:
a heating element adapted to heat a substance within a chamber, the chamber positioned and located proximate to the heating element;
an aeration chamber including at least one outlet, the aeration chamber in fluid communication with the chamber;
at least one vial in fluid communication with the aeration chamber; and
wherein first vapors from the chamber and second vapors from the at least one vial are mixed in the aeration chamber prior to being provided to the at least one outlet.

9. The vaporizer system of claim 8, wherein the heating element and the chamber are retained within a body of a portable unit.

10. The vaporizer system of claim 9, wherein the portable unit is adapted to be selectively coupled to a conduit that is in fluid communication with the aeration chamber.

11. The vaporizer system of claim 8 further including a tabletop unit with a body, and wherein the aeration chamber and the at least one vial are retained within the body of the tabletop unit.

12. The vaporizer system of claim 8, wherein each vial of the at least one vial further includes a mechanism that can be placed into an open position and a closed position.

13. The vaporizer system of claim 12, wherein when the mechanism is in the open position, the second vapors retained within the vial are placed into fluid communication with the aeration chamber.

14. The vaporizer system of claim 8, wherein each vial of the at least one vial includes an aperture that can be placed into an open position, a closed position, and at least one intermediate position between the open position and the closed position.

15. A system for vaporizing a substance comprising:
a unit including:
a body;
an aeration chamber comprising at least one outlet;
an at least one vial selectively removable from the body; and
a vial conduit coupling the aeration chamber and the at least one vial;
a portable unit including a chamber and a heating element; and
wherein when the portable unit is retained within the unit a portable conduit couples the portable unit to the aeration chamber such that the portable unit is in fluid communication with the aeration chamber.

16. The system of claim 15, wherein the at least one vial is adapted to selectively provide different amounts of aromatic vapors to the aeration chamber.

17. The system of claim 15, wherein each vial of the at least one vial further includes an adjustable opening that can be placed in an open position and in a closed position.

18. The system of claim 17, wherein the adjustable opening of each vial of the at least one vial can be selectively incremented between the open position and the closed position.

19. The system of claim 15, wherein each vial of the at least one vial further includes a mechanism with an aperture, and wherein a size of the aperture can be adjusted by a user.

20. The system of claim 15, wherein the system is in wireless communication with at least one remote device.
